(19) United States
(12) Patent Application Publication
(10) Pub. No.: US 2013/0130917 A1
(43) Pub. Date: May 23, 2013

(54) METHOD FOR SPECIFIC ENRICHMENT OF NUCLEIC ACID SEQUENCES

(71) Applicant: Wei-Wen Cai, Allen, TX (US)

(72) Inventor: Wei-Wen Cai, Allen, TX (US)

(73) Assignee: HALGEN CORPORATION

(21) Appl. No.: 13/349,718

(22) Filed: Jan. 13, 2012

Related U.S. Application Data
(60) Provisional application No. 61/432,432, filed on Jan. 13, 2011.

(51) Int. Cl. C12Q 1/68

(52) U.S. Cl. CPC C12Q 1/6874
USPC 506/2; 506/26

(57) ABSTRACT

The invention discloses a method for immobilizing nucleic acid probes to solid substrates. Also disclosed is a micro column format for specific sequence capture which enables efficient and convenient enrichment of target sequences from a complex source. The capture probes are immobilized onto microspheres or fibrous filter as the active component inside the column. The column format allows hybridization, post-hybridization wash and recovery of captured sequences all to take place in a simple device without sophisticated equipment.

### TECHNICAL FIELD

The present invention is in the technical field of genetic analysis. More particularly, the present invention is in the technical field of isolation of specific nucleic acid sequences from a complex source such as genome.

### BACKGROUND

Any biological systems, from freely living single cell bacteria to highly sophisticated multi-organ animals, at the molecular level, consist of thousands of basic elements such as genes and their products which interact in numerous synergetic ways to maintain the system's stability and the ability of the organisms to reproduce. To understand biological processes at the molecular level, we still rely on reductionist approaches which focus on a small subset of elements to study their basic function in the complex systems. Invariably, this involves isolation of basic elements from a complex source such as a genome or transcriptom for further analysis. For instance, to study how mutations of genes would lead to the development of cancer, we must isolate these genes from tumors and analyze their mutation spectra. Traditionally, gene sequences isolation was achieved through cloning of the respective genes. This is a very time consuming process. The development of polymerase chain reaction (PCR), which allows in vitro amplification of nucleic acid sequences, has dramatically reduced the efforts in disease gene identification. PCR amplification coupled with high-throughput DNA sequencing is a very powerful approach for genetic analysis. However, as the scale of analysis increases this approach becomes impractically expensive.

Technology development in recent years, especially high throughput DNA sequencing technologies, has sparked a revolution that will radically transform biological and biomedical research. It is increasingly realized that many biological and biomedical problems can and only be addressed through large scale sequencing of DNA or RNA. For example, through large scale sequencing, we can rapidly grasp the scale of mutations in cancers. Large scale and cost effective sequencing also makes previously difficult endeavors straightforward. For example, identification of a disease gene in a large genomic region can now be directly tackled by targeted DNA sequencing of the region harboring the disease gene. As these high throughput analysis technologies become increasingly accessible to researchers, they are frequently used to address previously impossible problems. However, broad applications of these technologies are still limited by their high costs in both equipment acquisition and reagent consumption. The cost of resequencing a mammalian-sized still remains in the range thousands of dollars, which is far too high for many applications that require sequencing of a large number of samples. A remedy for this is to target selected regions of interest for sequencing. This will require a step to specifically isolate the regions or specific set of gene targets of interest.

Capturing specific sequences from a genome or transcriptom is conceptually straightforward. Probes are designed to target those regions of interest. The targeted sequences are captured by hybridizing the probes to the targeted regions in a solution based or surface based hybridization format. In surface based hybridization format, probes are usually synthesis using in situ DNA synthesis approaches (See U.S. Pat. Nos. 8,058,004, 7,323,320, 7,183,406, 8,034,912, 6,586,211, 7,547,775) and the probes are arranged in an array format on a flat glass slide surface (See U.S. Pat. Nos. 6,600,031, 7,956,011, 7,291,471). The source sequences from which targeted sequences are to be captured are hybridized to an array of probes. Unhybridized sequences are removed by washing and the captured sequences are stripped off the array surface. This approach has some advantages in terms of flexibility of probes design and synthesis and convenience of use. However, major disadvantages of this approach include high cost of probe synthesis and low capture capacity of the array because of the limited amount of each probe achievable by in situ synthesis and the low efficiency of hybridization. Solution based hybridization format was developed to overcome these problems. In solution based sequence capture format, biotin labeled probes are usually used to hybridize to the source sequences such as a genomic fragment library in solution. The hybridization usually takes 48 to 72 hours. After hybridization, magnetic beads coated with strepavidin are added to bind to all the biotin labeled probes thus to separate the captured sequences from the unhybridized source sequences. The captured sequences are then amplified for sequencing. This approach has higher sensitivity than the surface based approach but reagent cost is still high. Limited capture capacity is the common limitation in current commercial kits for sequence enrichment. Therefore, there exists an unmet need for methods that have the advantage of high sequence enrichment efficiency, high enrichment capacity, high specificity and cost effective.

### SUMMARY OF THE INVENTION

The present invention provides methods for immobilizing nucleic acid probes to a solid substrate for capturing a desired set of sequences from a complex source such as genomic fragment library by hybridizing the source sequences to a solid substrate with immobilized nucleic acid probes that target to a large number of sequences of interest in source sequences. The hybridization is carried out in a convenient format to achieve high hybridization efficiency, high sequence enrichment capacity, and high specificity.

### BRIEF DESCRIPTION OF THE DRAWINGS

FIG. 1 depicts a micro spin column format for sequence capture. 1, microspin column; 2, ring fitting; 3, glass microfiber filter carrying nucleic acid probes; 4, nylon mesh disks; 5, microdropper bottom plug; 6, column cap; 7, syringe tube fitting to the top of the microcolumn; 8, flow control filter or membrane disk.

### DETAILED DESCRIPTION OF THE INVENTION

The process of conducting sequence selection from a complex source is disclosed in the present invention. The sequence enrichment method consists of four major steps:
1. Nucleic acid probes that target to specific sequences by hybridization are attached to an ensemble of micro particles or fibrous solid substrate.
2. The source sequence pool from which specific sequences to be enriched is hybridized to the probes attached solid substrate under optimal conditions for nucleic acid hybridization.
3. The unbound source sequences are separated from the solid substrate bound sequences.
4. The substrate bound sequences are released from the solid substrate.

The embodiments of the present invention are explained in detail hereafter.

### Nucleic Acid Probes

The nucleic acid probes of present invention include without limit oligonucleotides, purified cloned DNA from cDNA clones and genomic clones such as bacterial artificial chromosomes (BACs), fosmid clones, and a fraction of genomic sequences such as repetitive sequences like Cot I DNA.

Oligonucleotides are preferable 20 to 100 base pairs in length. More preferably, Oligonucleotides are concatenated to larger polynucleotides, preferably to 400-1200 bases in length.

Oligonucleotide probes can be made by standard oligonucleotide synthesis methods. Preferably, they are synthesis in parallel by in situ surface synthesis in an array format (See U.S. Pat. Nos. 6,600,031, 7,956,011, 7,291,471). The oligonucleotides are designed in such a way so that each probe is flanked by a universal sequence at one end and a different universal sequence at the other end. The oligonucleotides are then stripped off the surface as a pool by 0.05-0.1 M NaOH and amplified by polymerase chain reaction using a suitable pair of primers targeting to the flanking sequences of each oligonucleotide probes.

Purified cloned DNAs such as cDNA clones, genomic clones such as BACs, yeast artificial chromosomes, fosmid clones, by normal preparation procedure, usually are longer than 500 bases in size. These clones DNAs can be deposited to substrate surfaces carrying an expoxide functional group and effectively immobilized on the surface. Alternatively, these cloned DNAs may be chemically modified to carry a functional group that is specific to substrate surfaces such as a glass surface (See U.S. Pat. Nos. 6,048,695, 6,858,713, 6,979,728).

### Substrates for Nucleic Acid Immobilization

Nucleic acid can be immobilized on various substrate surfaces including, though not limited to, glass, quartz, mica, carbon, apatite, alumina, silica, silicon carbide, silicon nitride, boron carbide, graphite, polycarbonate, polypropylene, polyamide, phenol resin, epoxy resin, polycarbodiimide resin, polyvinyl chloride, polyvinylidene fluoride, polyethylene fluoride, polyimide, acrylate resin, and so forth.

The substrate can be in various shapes and sizes, including microspheres, flat surfaces, fibers, filter disks with straight through channels, and so forth. In the present invention, substrates with large surface areas are preferred. Such substrates include, not limited to, microfibers, porous glass microspheres, microbeads, ceramic filters, and so forth.

### Linker for Coupling Nucleic Acid Probes to Substrate

The present invention prefers the use of substrates with large surface areas, which will make it difficult to synthesize a large number of probes of different sequences directly on the substrate surfaces. The nucleic acid probes must be made separately and then immobilized to the substrate. While there exist various methods for coupling nucleic acid probes to a solid substrate, few can be directly utilized for the purpose of present invention which demands high probe capacity, high hybridization efficiency and low nonspecific hybridization background, and the ability to sustain stringent post hybridization washes.

The present invention provides methods for coupling nucleic acid probes through a linker attached to the solid substrate. The linker serves two purposes: coating the surface to turn the surface into negatively charged so that nonspecific absorption of nucleic acid is eliminated, thus reducing the hybridization background, and the linker keeps the coupled nucleic acid probes away from the solid surface to increase the hybridization efficiency. In the present invention, the preferred linker is attached to the substrate at the 5′ end. Preferred linkers are oligonucleotides with a phosphate or amine group at the 5′ end.

### Immobilizing Linker to Substrate

Two preferred methods are used in the present invention to immobilize the linker to the substrate.

Method A. Substrate surface is first coated to contain primary amine groups. Surfaces containing silanol groups such as glass surfaces, silica surfaces can be modified by silane compounds with an amine end group. These silanes include 3-aminopropyltrimethoxysilane, 3-aminopropyltriethoxysilane, 4-aminobutyltriethoxysilane, aminopropylsilanethriol, and so forth. Oligonucleotide linkers with a 5′ phosphate group can be conjugated to the amine coated surfaces by forming phosphoramidate linkage between the phosphate group and the amine group. This conjugation reaction is mediated by a water-soluble carbobiimide such as 1-ethyl-3-(3-dimethylaminopropyl)carbodiimide (EDC).

Method B. Substrate surface is first functionalized to contain carboxylate groups. Surfaces containing silanol groups such as glass surface, silica surfaces can be readily functionalized to contain carboxylate groups by treating the surfaces in an aqueous solution of 10-50 mM sodium carboxyethylsilanethriol. Oligonucleotide linkers with a 5′ amine group can be coupled to the carboxylate coated surfaces through carbobiimide mediated amide bond formation reaction in the presence of EDC.

Coupling Nucleic Acid Probes to a Substrate with an Immobilized Oligonucleotide Linker

The nucleic acid probes are coupled to a substrate indirectly through an immobilized oligonucleotide linker using one of two methods depending on the nature of nucleic acid probes to be immobilized.

Method A. Nucleic acid probes are conjugated to the immobilized oligonucleotide linker by ligation. This method of conjugation is preferably applied to single stranded oligonucleotide probes. The ligation is mediated by a helper primer which is complementary to the 3′ end of the linker and to the 5′ end of the oligonucleotide probes. The helper primer hybridizes to the linker and the oligonucleotide probes to bring them in close proximity that allow the ligation to happen. In the same ligation reaction, the oligonucleotide probes may be further concatenated into longer probes with the assistance of another helper primer that bridges the oligonucleotide probes.

Method B. The nucleic acid probes are effectively coupled to the linker by using the linker as a primer to copy the sequences of nucleic acid probes in a reaction containing DNA polymerase and dNTP in a proper buffer. This method is preferably applied to couple probes prepared by in situ surface synthesis. The probes contain universal adaptor sequences at both ends which are used for PCR amplification. The coupling can be achieved by a simple primer extension reaction. But preferably, thermal cycling is used to increase the efficiency of coupling reaction.

Capturing Specific Sequences from Source Sequences

Complex genomes or transcriptoms of selected tissues are two major sources of sequences from which a subset of sequences are captured for further analysis such as sequencing. In applying the method of present invention to enrich specific sequences from a genome, genomic DNA is used and fragmented to the size of 100 bp to 500 bp by one of these methods: sonication, nebulization, chemical treatment such as heating in NaOH solution, or enzymatic digestion such as treating with DNase or restriction enzyme digestion. In applying the method to sequence transcriptomes, the RNA is first converted into DNA by reverse transcriptase under proper conditions. The product DNA can be further fragmented by methods as described for genomic DNA.

The fragmented DNA is purified by ethanol precipitation, gel filtration, ion exchange chromatography. The purified DNA is then dissolved or diluted in a proper reaction buffer with Klenow enzyme and dNTP to repair the fragment ends. The end repaired DNA is ligated to two adaptor oligonucleotide sequences. PCR (polymerase chain reaction) is used to amplify the adaptor ligated population.

The amplified fragment population is denatured by heating to an elevated temperature in a hybridization buffer containing 0-50% of formamide, 3-12% of dextran sulfate or polyethyleneglycol, 1-6% of sodium dodecyl sulfate, 0.3 M NaCl, 20 mM sodium citrate at pH7.5. Hybridization is carried out at 37-65° C. depending on the stringency requirements which depend on the application.

The unbound source sequences in the solution can be simply washed away. However, the non-specifically bound sequences must be removed under conditions depending on the applications. For example, if the method is used to capture the exonic sequences from a tumor sample for sequencing to detect mutations in cancer related genes relatively low post hybridization wash stringency should be used to avoid loss of mutated sequences. The stringency of hybridization can be controlled by wash temperature and the salt concentration in the wash solution. Typically, the wash temperature is set at 57° C. and the wash solution contains 0.1-0.3 M NaCl.

The enriched sequences can be released from the solid substrate by one of these two methods: Heating to 100° C. for 5 minutes in TE buffer (10 mM Tris.HCl, pH=7.6, 1 mM EDTA). Or using 10-30 mM of NaOH or KOH to strip for 2 minutes.

### Hybridization Format for Sequence Capture

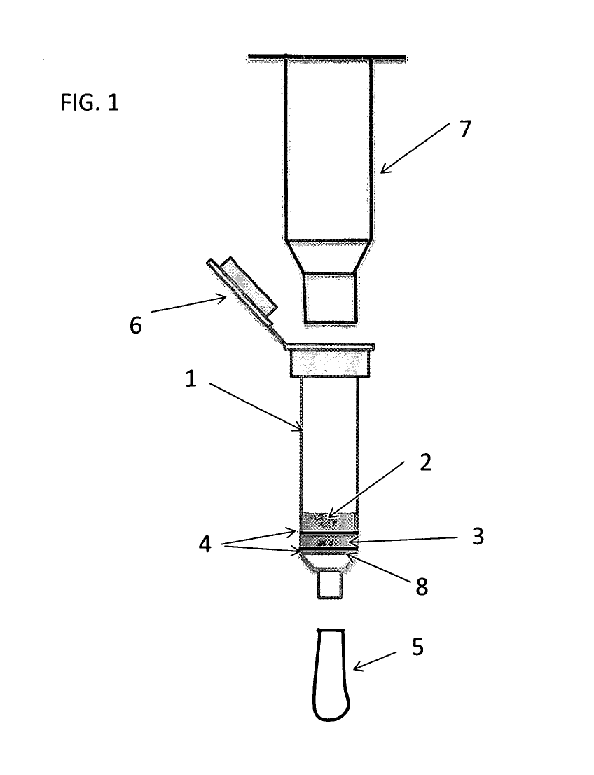

The present invention provides a convenient format to carry out sequence capture hybridization. The device setup is depicted in FIG. 1. In a preferred embodiment, the sequence capture substrate, which is made of glass microfiber filter with covalently bound nucleic acid probes, is sandwiched by two thin layers of mesh, preferably nylon mesh, and the sandwich is locked in place to the bottom of a microspin column by a tiny ring perfectly fitting to the inside of the microcolumn.

During hybridization reaction, the bottom plug is attached to the bottom of the microspin column and the cap is also snapped to the top to prevent loss of liquid inside the column. The bottom plug is a small rubber dropper head. Preferably, the microspin column is placed in a device that occasionally squeezes the dropper head to generate agitation in the hybridization solution to increase the hybridization efficiency. Alternatively, agitation can be effectively attained by placing a small solid bead inside the microspin column which is rotated slowly in a hybridization incubator.

The microspin column has a standard dimension of 8-9 mm in diameter and 25-29 mm in length. The column fit into a standard 1.5 or 2.0 mL microcentrifuge tube. After hybridization, the column is washed by plugging in a syringe tube to the top of the column. Wash solution in the syringe tube drips down to the column to constantly supply fresh wash solution to the substrate. The flow rate of wash solution through the substrate is controlled by a filter or membrane at the bottom of the column. The setup is in essence an automatic washing device which requires no additional equipment such as pump or shaker to agitate the wash solution for effective washing.

### Sequence Capture to Reduce Unwanted Sequences in Nucleic Acid Samples

Highly expressed genes are defined as those genes that expresses 10 times above the median level of all the genes expressed in a selected tissue. These genes produce highly redundant sequence reads which significantly consume the sequencing capacity. In the worse cases, these highly expressed genes may mask the detection of mutations in genes expressed at a low level. Traditional protocol of normalization to even out the expression level of genes in a transcriptom involves a lengthy and expensive process and is now rarely practiced. The present invention provides an excellent approach to this problem.

The present invention also offers a convenient way to remove repetitive sequences from a complex genome. Repetitive sequences account for about 50% of the human genome. In a whole genome sequencing project using the current generation of sequencing technologies which typically provide short reads of 75-150 bases, more than half of the reads can not be mapped back to the genome due to the presence of repetitively sequences. Removal of repetitive sequences will increase the number of usable reads at the same sequencing redundancy.

### EXAMPLES

### Example 1

### Preparation of Aminated Glass Microfiber Filter Disks

Glass microfiber filters were purchased from VWR. The filters were cleaned by soaking in 3M HCl overnight. Acid was rinsed off by distilled water and the filters were dried at 65° C. for 20 minutes. Filters were cut into small disks using a paper punch. The filters disks were aminated by treating at 65° C. for 16-20 hours in 10 mM 3-aminopropyltrimethoxysilane in 50% ethanol. Treated disks were rinsed by distill water two time and air dried on a piece of clean paper towel at room temperature.

### Example 2

### Preparation of Carboxylated Glass Microfiber Filter Disks

The microfiber filters were cleaned by soaking in 3M HCl overnight. Acid was rinsed off by distilled water and the filters were dried at room temperature overnight. Filters were cut into small disks using a paper punch. The filters disks were carboxylated by treating in 50% ethanol containing 15 mM of sodium carboxylethylsilanetriol at room temperature for 20-24 hours. Treated disks were rinsed by distill water two time and air dried on a piece of clean paper towel at room temperature.

### Example 3

### Immobilizing 5′ Aminated Linker to Carboxylated Glass Microfiber Filter Disks

Place one carboxylated glass microfiber filter disk prepared by procedure described in Example 2 in a well of flat bottom 96 well microplate. Add 20 μl 5′ aminated linker at 0.2-0.5 μg/μl in 0.1 M imidazole, pH6.0. Add 5 μl of 0.2 M carbodiimide EDC in DMSO. React at 37° C. for 60-120 minutes. Wash the filter disk with distilled water two times. Wash the filter disk with 100% ethanol once and air dry the filter disk in the microplate well.

### Example 4

### Immobilizing 5′ Phosphorated Linker to Aminated Glass Microfiber Filter Disks

Place one aminated glass microfiber filter disk prepared by procedure described in Example 1 in a well of flat bottom 96 well microplate. Add 20 μl 5′ phosphorated linker at 0.2-0.5 μg/μl in 0.1 M imidazole, pH6.0. Add 5 μl of 0.2 M carbodiimide EDC in DMSO. React at 37° C. for 2-3 hours. Wash the filter disk with distilled water two times. Wash the filter disk with 100% ethanol once and air dry the filter disk in the microplate well. Add 50 μl 1 M succinic anhydride in DMSO to the well and react at room temperature for 30 minutes. Remove solution by aspiration. Add 100 μl distilled water to rinse the filter 3 times. Air dry the filter disk at room temperature.

### Example 5

### Preparation of Exomic Probes

Probe sequences were downloaded from public genome databases. All probes are designed to have an annealing temperature of 60° C. Probes sequences were flanked by a pair of adaptor with sequences: Adaptor1, with sequence of CCTCGTCCACGGCTC at the 5′ end Adaptor2, with sequence of AGGGTCGGCACGGTT at the 3′ end. The probe sequences were sent to a commercial supplier to synthesize oligonucleotides on a microarray by in situ synthesis method. After receiving the microarray containing the oligonucleotide probes, we stripped off the probes by spreading 0.5 M NaOH on the microarray. Stripping reaction took 2 hours at room temperature. Probes solution was collected and dialyzed against TE buffer on a piece of dialysis membrane which was place on top of a gel with TE buffer. Dialysis lasted 16-20 hours at room temperature. Probe solution was transferred to a 1.5 ml microtube. A portion of probe solution which contain about 200-500 copies of each probes was taken for PCR to amplify the probes. Amplification was carried in 100 μl reaction containing 50 mM Tris pH 8.2, 100 mM KCl, 1.5 mM MgCl₂, 0.1% Triton X-100, 10 units of Taq polymerase, 0.2 mM dNTP, 0.5 uM forward primer: Fprimer1, with sequence of CCTCGTCCACGGCTC, 0.5 uM reverse primer: Rprimer1, with sequence of AACCGTGCCGACCCT. Thirty cycles of PCR was carried out using this thermal cycling program: 92° C. for 20 seconds, 53° C. for 30 seconds, and 65° C. for 25 seconds. This primary PCR reaction product is the exomic probe pool containing all the designed exomic sequences. The exomic probe pool was the seed for further expansion. This seed probe pool was stored at −20° C. without purification. Expansion of primary probe pool was carried out in a 96 well PCR plate. Each well is filled with 100 μl PCR reaction using 0.1 μl of primary probe pool as the template for amplification. After 30 cycles of PCR amplification solution in all 96 wells is pooled in a reagent tray and NaCl was added to 0.5 M. Ethanol precipitation was used to purify the amplified genomic probes, which subsequently was dissolved in TE buffer at 0.1-0.3 μg/μl.

### Example 6

### Preparation of Glass Microfiber Filter Column for Capturing Exomic Sequences in Genomic DNA

5′ phosphorated linker, Linker1 with sequence, ACTATCCTCGTCCACGGCTC, was coupled to glass microfiber filter disks using the protocol described in Example 4. Two 8.5 mm filter disks in diameter was placed in a 0.5 mL PCR tube on ice. 200 μl of PCR reaction mix containing 50 mM Tris pH 8.2, 100 mM KCl, 1.5 mM MgCl₂, 0.1% Triton X-100, 20 units of Taq polymerase, 0.2 mM dNTP, 0.5 uM reverse primer, Rprimer2, with sequence of AACCGTGCCGACCCT, and 2 μg of exomic probe DNA prepared using protocol described in Example 5 was added to the PCR tube containing filter disks. Place the tube in a 48 well PCR machine that accepts 0.5 mL tube and start the PCR for 35 cycles using a program as: 94° C. 35 seconds, 52° C. 60 seconds, and 72° C. 30 seconds. After PCR, remove the reaction solution, add 300 μl TE buffer to the tube and heat tube on a 100° C. heat block for 5 minutes. Remove the TE buffer by aspiration. Rinse the filter disks in a 100 mm petri dish containing 30 mL TE buffer. Air dry the filter disks on a piece of Whatman paper. Load the filter disks to a microcolumn as depicted in FIG. 1.

### Example 7

### Preparation of Genomic Fragment Library

10 μg of human genomic DNA was sheared by sonication to 300-500 bp. A library of adaptor ligated fragments was prepared for the sheared DNA using commercial kits and following the protocols provided by the supplier.

### Example 8

### Capturing Exonic Sequences from Genomic DNA

Use 0.5-1.0 μg of adaptor ligated genomic DNA prepared in Example 7 for exonic enrichment. Mix the adaptor ligated genomic DNA with 30 μg of human Cot I DNA and 25-50 μg of linker block oligos mix which has the same sequence in double stranded form as the flanking adaptors on the ligated genomic DNA. Add NaCl to a final concentration of 0.5 M. Add equal volume of isopropanol and mix by vortexing. Spin at 14,000 rpm for 6 minutes. Remove solution by aspiration. Rinse the pellet with 80% of ethanol. Remove ethanol by aspiration. Dry the pellet in the 47° C. circulating incubator for 5 minutes. Dissolve the pellet in 20 μl TE buffer. Add 40 μl of hybridization buffer of composition: 30% formamide, 2×SSC (pH7.20), 6% SDS, 10% dextran sulfate. Mix by pipetting and then vortex briefly. Denature the probe mix on a 100° C. heating block for 3 minutes. Transfer the probe solution to a 47° C. incubator and incubate for 15 minutes. Transfer the probe solution to the filter disks in the exome capture column as described in Example 6. Place a glass bead 2-3 mm in diameter inside the capture column. Close the lid tightly and place the column inside a 15 mL tube. Use some cotton to hold the column to the bottom of the tube and tighten the screw cap. Fix the tube in a position perpendicular to the rotation axis in a hybridization 47° C. incubator rotating at 6 turns per minutes and hybridize for 16 to 20 hours. After hybridization, remove the bottom plug and place in a 1.5 mL tube. Spin for 1 minute at 14,000 rpm to remove the hybridization solution. Place the column back in the capless collection tube and pipette in 450 μl 1×SSC with 0.1% Triton X-100. Plug in the 5 mL BD syringe tube to the column and pour in 5 mL of 1×SSC with 0.1% Triton X-100. Place the column in one of the hole on the lid of the collection container and let the wash solution drain in a 57° C. incubator through the column completely. Fill up the syringe tube with TE buffer (pH=7.9) with 0.1% Triton-X 100 and let the buffer drain through at 37° C. Remove the syringe and place the column back in the collection tube. Spin at 14,000 rpm for 1 minutes. Aspirate to remove solution in the collection tube. Add 350 μl of TE buffer and spin briefly to rinse the column. Repeat this step two times. Spin the collect at 14,000 for one minute. Place the column on a new 1.5 mL tube. Add 60 μl TE buffer to the column. Gently tap the column to help spread out the solution. Incubate the column in a 110° C. incubator for 5 minutes. Spin to collect the solution with stripped off captured exonic sequences.

### Example 9

### Assay to Evaluate the Capture Efficiency of Sequence Capture Microfilter Column

To evaluate the capture efficiency of sequence capture microfilter column we used fluorescently labeled genomic DNA prepared as follows: 0.1 μg of human genomic DNA is amplified and amine labeled in a 100 μl reaction using a Radprime random priming kit purchased from Invitrogen. The labeling reaction was supplied with 0.15 mM of AA-dUTP to replace 70% of dTTP in a normal reaction containing 0.2 mM dTTP, 0.2 mMdCTP, 0.2 mM dATP, 0.2 mM dGTP. Forty units of Klenow exo-enzyme was added to the reaction mix which was incubated at 37° C. for 3 hours. Purify the amplified product by standard ethanol precipitation. About 5-6 μg of amplified and labeled genomic DNA was recovered after ethanol precipitation. The amine labeled products was dissolved in 0.1 M NaHCO₃ buffer, pH9.7. Add 3 μl of cy3 NHS amine reactive dye solution and mix. React at room temperature for 2-3 hours. Purify the now cy3 label genomic DNA by ethanol precipitation. Dissolve the pellet in 500 μl TE buffer and purify again by ethanol precipitation. Dissolve the cy3 label genomic DNA at 0.1 μg/μl in TE buffer. Use 2.5 μg cy3 label genomic DNA to set up hybridization reaction to capture exomic fragments following the procedures described in Example 8. The enriched exomic sequences was collected and compared to a series of original input cy3 labeled genomic DNA to estimate the concentration of enriched exomic sequences using the procedure as follows: The solution of enriched exomic sequences was spotted on an aminated glass slide surface together with serial dilution samples of the original genomic input DNA. Dry the spots at 65° C. for 10 minutes. Rinse the slides with 50% ethanol and dry it again at 65° C. for 10 minutes. Image the surface using a microarray scanner. Quantify the amount of fluorescence of all the spots. For negative control to evaluate the level of nonspecific absorption of the sequence capture column same capture reaction was performed in parallel but using a microfilter column without attached exomic capturing probes. Over 10 capture reaction was performed. The results showed that about 1-3% of the target sequences could be enriched by the capture microfilter column. The level of nonspecific absorption was estimated to be less than 3% of level of enriched sequences.

### Example 10

### Preparation of Microfiber Filter Column for Removal of Repetitive Sequences in Genomic DNA

15 μg of human Cot I DNA was directly immobilized on 3 glass microfilter disks of 8.5 mm in diameter using procedure described in Example 4. The filter disks were made into a sequence capture column as depicted in FIG. 1. 2 μg of sheared human genomic DNA was hybridized to the Cot I DNA capture column following the procedure described in Example 8, except that Cot I DNA was not used to block the repeats in genomic DNA. The hybridization buffer was collected to evaluate the effect of removal by the Cot I DNA capture column. Genomic DNA in the hybridization buffer was recovered and purified further by ethanol precipitation. 0.1 μg of such recovered DNA was fluorescently labeled in two colors cy3 and cy5 using the procedure described in Example 9. In parallel, 0.1 μg of original sheared genomic DNA was labeled in cy3 and cy5 following the same procedure. The effect of repeat reduction in genomic DNA by the Cot I DNA capture column is evaluated by the following assay: Mix 50 ng of cy3 labeled recovered DNA with 50 of cy5 labeled original sheared genomic DNA and hybridize to a BAC clone microarrays which contain clones of variable amount of repeats in discrete spots in a proper microarray buffer for 3 hours at 37° C. After the hybridization, use distilled water to rinse of all residual hybridization solution and dry the array by blowing compressed air to the array. Image the array in both cy3 and cy5 channel. The effect of repeat removal is shown by significant loss of signal in the cy3 channel. This effect is confirmed by the following assay: Mix 2 μg cy3 labeled recovered genomic DNA and 2 μg cy5 labeled original sheared genomic DNA and 50 μg Cot I for repeat suppression. The probe mix is hybridized to a BAC clone array in an array hybridization buffer at 37° C. for 16 hours. Rinse off the hybridization solution and image the array after drying. The effect of repeat removal is indicated by the significantly higher signal level in the cy3 channel compared to that of cy5 channel due to the fact that in equal amount of labeled probe the recovered genomic DNA contains more unique sequence than the original genomic DNA. The above assay results were further confirmed by cy3/cy5 dye swap experiments.

### Example 11

### Normalizing the Copy Number of Highly Expressed Genes in Lung Tumor Tissue Using Microfiber Filter Column

Highly expressed genes in lung cancer samples were identified from expression data in the public database. Genes with an expression level 10 times above the median levels of all expressed genes in lung tissue are regarded as highly expressed. The exonic sequences of these genes were obtained from public genome databases. Sequence capture probes were designed, probes were synthesized, microfilter columns were prepared following procedures described in Example 5 & 6. Commercial kits were used to prepare cDNA from RNA isolated from lung tumor samples. The lung tumor cDNA was labeled in cy3 and a normal control cDNA sample was labeled by cy5 using commercial kits. Gene expression microarrays containing oligonucleotide probes from 100 highly expressed genes and control probes from 100 genes expressed at median level were custom made using procedures described in Example 4 &6. 2 μg of cy3 labeled lung tumor cDNA (see above) was mixed with hybridization buffer and hybridized to the prepared microfilter columns at 37° C. for 3 hours. The hybridization buffer was collected and mixed with 2 μg of cy5 labeled normal control cDNA and hybridized to a gene expression array described above. For control, 2 μg of cy3 labeled lung tumor cDNA was mixed with 2 μg of cy5 normal control cDNA and hybridized to a separate expression array. The relative fluorescence intensities of cy3 and cy5 channels for each gene were compared for the two array hybridizations. On average, the level of highly expressed genes could be reduced to within 3 folds above the median level after the cDNA was normalized through hybridization to the microfilter column.

### CONCLUSION

The advantages of the present invention include, without limitation, that the invention has overcome many limitations of the existing methods for sequence enrichment. The existing methods for sequence capture either require expensive equipment or involve very complicated procedures. Sequence capture kits based on the present invention have such high capacity that sequence capture from a multiplex sample pool becomes possible. The kits also have very high capture efficiency to make it possible to eliminate some amplification steps in the current targeted sequencing protocols. The high efficiency of capture and the micro column capture format require significantly less amount of input source sequences, making it less challenging to capture target sequences from a rare source.

In broad embodiment, the present invention is a novel micro column format sequence capture method based on the principle of exceedingly reducing the ratio of liquid phase to surface area of the probe attached substrate to dramatically increase the hybridization efficiency. This format successfully overcomes the limitations of the existing microarray based and solution based sequence capture methods, while retaining the ease of manipulation of the microarray based method and the high efficiency of the solution based method. In one embodiment, the method can be used to specifically remove unwanted sequences from a plurality of nucleic acid. For example, repetitive sequences in the human genome occupy about 50% of the total DNA. When these repetitive sequences are reduced to a substantially low level the content of specific sequence information will be significantly increased, thus reducing the sequencing cost by almost half. Because of these advantages, the present invention has broad utilities in a variety of applications in research and clinical diagnosis.

While the foregoing description of the invention should enable one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

Sequence Listing
Primer and oligonucleotide sequences:

| | |
|---|---|
| Adaptor1 | CCTCGTCCACGGCTC |
| Adaptor2 | AGGGTCGGCACGGTT |
| Fprimer1 | CCTCGTCCACGGCTC |
| Rprimer2 | AACCGTGCCGACCCT |
| Linker1 | ACTATCCTCGTCCACGGCTC |
| Rprimer2 | AACCGTGCCGACCCT |

We claim:

1. A method for immobilizing nucleic acid probes to a solid substrate such as glass microspheres, glass microfibers, and so forth comprising: reacting the surface of said solid substrate with a silane solution of 1 to 100 mM 3-aminopropyltrimethoxysilane or 1 to 100 mM sodium carboxyethylsilanetriol to produce a amine or carboxylate functionality respectively on the surface of said solid substrate; coupling said amine or carboxylate functionality to the respectively 5′ phosphate or amine end of an oligonucleotide linker in a solution of 1-ethyl-3-(3-dimethylaminopropyl)carbodiimide; linking the 3′ end of said oligonucleotide linker to said nucleic acid probes.

2. The method of claim 1, wherein: the method of linking the 3′ end of said oligonucleotide linker on said solid substrate to said nucleic acid probes comprising: ligating the 3′ end of said oligonucleotide linker to said nucleic acid probes using a DNA ligase in a reaction solution containing a helper primer which is complementary to a portion of the 5′ end of said nucleic acid probes and complementary to a portion of the 3′ end of said oligonucleotide linker on said solid substrate to bring the 5′ end of said nucleic acid probes to the 3′ end of said oligonucleotide linker into close proximity for ligation reaction mediated by T4 DNA ligase.

3. The method of claim 1, wherein: the method of linking the 3′ end of said oligonucleotide linker on said solid substrate to said nucleic acid probes comprising: exposing said solid substrate containing said oligonucleotide linker to a solution containing said nucleic acid probes which contain a portion of sequence at the 3′ end complementary to the 3′ end of said oligonucleotide linker, performing thermal cycling in a solution containing thermal stable DNA polymerase, dNTPs and necessary components that support polymerase chain reaction, thereby copying the sequences of said nucleic acid probes to the 3′ end of said oligonucleotide linker.

4. A method for extracting specific nucleic acid sequences from a sequence source, comprising: contacting said sequence source with a glass microfiber substrate containing a plurality of immobilized nucleic acid probes of 20 to 2,000 nucleotides that hybridize to their respective specific complements in said sequence source such as a library of fragments from a genome or a transcriptom in a hybridization solution containing 0-50% formamide, 10-12% dextran sulfate, 1-6% sodium dodecylsulfonate, 0.9M NaCl, 50 mM sodium citrate at pH7.3, removing said hybridization solution and washing said solid substrate free of unbound source sequences by eluting the said substrate with necessary amount of a buffer containing 10-20 mM Tris-HCl, pH7.5 and 0.1-0.5% TritonX-100 at 45-50° C. in an incubator for 30-60 minutes, separating the sequences that hybridized to said immobilized probes on said solid substrate from said solid substrate by stripping the said substrate in 5-10 mM Tris-HCl, pH7.5 at 80-90° C. for 10-20 minutes, thereby isolating the desired sequences from said sequence source.


SEQUENCE LISTING (5 sequences)

SEQ ID NO: 1 - DNA, 15 bp, Artificial
  Synthetic oligonucleotide
cctcgtccacggctc

SEQ ID NO: 2 - DNA, 15 bp, Artificial
  synthetic oligonucleotide
agggtcggcacggtt

SEQ ID NO: 3 - DNA, 15 bp, Artificial
  synthetic PCR primer
cctcgtccacggctc

SEQ ID NO: 4 - DNA, 15 bp, Artificial
  synthetic PCR primer
aaccgtgccgaccct

SEQ ID NO: 5 - DNA, 20 bp, Artificial
  synthetic oligonucleotide
actatcctcgtccacggctc